(19) United States
(12) Patent Application Publication
(10) Pub. No.: US 2015/0374220 A1
(43) Pub. Date: Dec. 31, 2015

(54) METHOD FOR CLEANING A MIRROR WITH HANDLE FOR MEDICAL OR DENTAL EXAMINATION DURING USE BY MEANS OF A COMPRESSED AIR FLOW AND FOR THIS METHOD CUSTOMIZED MIRROR WITH HANDLE

(71) Applicant: Maarten Antoon Nicolaas DEN HARTOG, (US)

(72) Inventor: Maarten Antoon Nicolaas Den Hartog, Arnhem (NL)

(21) Appl. No.: 14/762,836

(22) PCT Filed: Mar. 11, 2014
(86) PCT No.: PCT/IB2014/000661

§ 371 Date: Jul. 23, 2015

(30) Foreign Application Priority Data
Mar. 12, 2013 (NL) 1040090

(51) Int. Cl. A61B 1/12; A61B 1/253; A61B 5/00; B08B 5/02

(52) U.S. Cl. CPC A61B 1/126; B08B 5/02; A61B 1/253; A61B 5/0079

(57) ABSTRACT

Method for cleaning of a mirror with handle for medical or dental examination during use and a specifically for this method customized mirror with handle for medical or dental examination, consisting of the combined use of a hydrophobic or super-hydrophobic, and non-stick reflecting surface and the controlling of an compressed air flow with an increased downward pressure in an angle of 5 to 25 degrees over this reflecting surface, by use of diverging control surfaces in the air duct.

Problem with the use of mirrors for medical or dental examination is that during use the mirror surface is studded with dirt and moisture, through which the sharp image reflection is reduced or even completely vanished.

This problem is described in various patent applications.

The many technical solutions that have been put forward for this problem in all cases lead to the situation that the mirror or the related elongate handle increase in size and therefore provide less freedom of movement than the known dental mirrors without those improvements. In addition those technical solutions result in more complex shapes, which are more difficult to clean or sterilize, and more difficult to produce and therefore are more expensive.

Examples of this are U.S. 2004/0076019 and EP 2181643 in which a rotating mirror with an extraction system is described; U.S. Pat. No. 3,969,824, in which a fluid supply and an extraction system is described, as well as the suggestion is made to create a hydrophilic surface by means of an additive; U.S. 2010/0261132 in which a combination of compression of air and spraying liquid over the reflecting surface is described, preferably by adding of an additional substance to the liquid, through which the hydrophobic effect on the mirror reduces in order for the fluid to more easily glide over the surface.

In the prior art, a method is also known in which a mirror surface is cleaned just by an air flow. This can be achieved, as evidenced by U.S. 2005/0282103 and U.S. 005449290 through a more simple instrument. In practice, however, this method is insufficient, because the majority of the dirt is sticking to the surface and the air flow has insufficient control over it. Examples of these are U.S. Pat. No. 5,449,290 and US2005/0282103. Known are also solutions, where only air is extracted at the periphery of the mirror surface in order to remove moisture and dirt from the reflecting surface.

For example, see in U.S. Pat. No. 8,133,052. This solution does not work sufficiently either for the same reason as in the sole air flow over a surface.

The best given working methods in the prior art are those in which both air and liquid are blown on the reflecting surface and are eventually also extracted again, whereby it is proposed to add substances to the liquid, in order to reduce the hydrophobic effect on the reflecting surface or, in other words spreading out the fluid more evenly over the mirror surface. The latter shows that the principle without this addition does not work completely satisfactorily.

Apart from this application, in the state of the art there are known methods for creating hydrophilic glass surfaces by using for example polymeric coatings in order to prevent fog formation. For example, in DE 2051832 and DE 2051832 or 20040107976 KR.

The hydrophilic effect has the result that the liquid with dissolved therein the dirt as will deposit as a film on the mirror surface. The fact that the fluid adheres better to the surface of the mirror through the hydrophilic character of the reflecting surface, or by adding substances to the liquid, may have the advantage, that dirt that is excited on the surface will more readily soluble, but it also has the disadvantage of the necessity of a stronger air and fluid flow in order to blow off the liquid from the surface than in the situation when no provisions were made to create a hydrophilic surface. In the latter case the deposited dirt on the mirror surface will dissolve less easy in the liquid. A strong and therefore more noticeable air flow or fluid flow is a nuisance in the treatment, both for the patient and the attending physician.

It is the goal to create a method for cleaning of a mirror surface of a mirror of the above-mentioned type, wherein a small i.e. not noticeable flow of air is sufficient to keep free the mirror surface from moisture and dirt as effective and possibly more effective as is the case with all the methods of the up to now known solutions, as well as to create a mirror of this type, in which the principles of this method have been incorporated, or are applied to and that, if possible, is as good or even better to handle than the up to now known traditional type, without those cleaning facilities.

In order to meet this goal the invention proposes to not create a hydrophilic surface, but instead of this to create a surface that is super hydrophobic, non-stick and anti-bacterial, through which in the first place dirt and moisture can not stick to the surface and in the second place through which the moisture deposit on the surface will draw together in little balls and a relative large contact angle is formed, and because of these two effects it is not only possible that one airflow over the mirror surface is sufficient to free the surface of dirt and moisture but also that the air flow can be limited both in strength and in size and it is no longer necessary to make use of other ways i.e. water to keep the mirror surface free from moisture and dirt causing the possibility to significantly reduce dimensions of the handle and nozzle.

A super-hydrophobic nature of the reflecting surface can be obtained by mounting solid hydrophobic nano-particle on to the reflecting surface. The process of a creating surfaces that are super hydrophobic in itself is a known process. For this purpose a suitable technique is, for example, the application of dissolved glass particles in liquid and applying this to the reflecting surface. After the liquid has evaporated, a layer remains, changing the surface into a harder, hydrophobic, non-stick and anti-bacterial surface. This coating layer is non-removable and resistant to high temperatures and a variety of aggressive chemicals. Another technique to create hydrophobic surfaces is plasma coating, wherein the objects to be coated are placed in a plasma machine and hydrophobic particles are projected on the surface in such a way that the surface becomes a hydrophobic and non-stick nature of being . Yet another technique consists of applying a layer of hydrophobic polymer. Examples of this are poly-tetra-fluor-ethylene (PTFE), perfluoraloxypolymeren (PFA), fluorinatedethylenepropylene (FEP), and polydimethylsiloxane (PDMS).

For the purpose of the target the invention further provides a combined improvement of effect of both the air flow and the functionality of the mirror, by using a triangular shaped mirror and by not casing the reflecting surface of the mirror from the place of connection of the mirror and handle in all directions of the mirror surface and by steering the air flow from above this mounting point through an in or by the hollow handle created air duct in a gentle angle of 5 to 25 degrees, by means of an air compression arranged by an narration of the air duct, resulting in an air flow of a higher pressure than the original supply pressure over the mirror surface and to enable this air flow to diverge by means of among a variety of angles placed upright surfaces in the air duct, in such a way that the air flow is substantially distributed over the surface of the triangular shaped mirror surface, and in such a way that the entire air flow is flowing under effectively downward pressure over the reflecting surface, whereby the triangular mirror shape also offers the possibility to place instruments like i.e. a drill, dental probe or tweezers next to the side of the mirror and then are observably reflected in the rear portion of the triangular reflecting surface.

By pressing the air flow under the said angle on the reflecting surface, the air flows with further downward pressure over the entire surface. By compressing the air just before the opening of the air outlet it will flow stronger out of the air outlet and thereby retains sufficient strength to blow clean the whole surface including the borders of the reflective surface.

Medical instruments must be sterilized effectively. In the case that an instrument contains inaccessible or difficult accessible places or obstacles this may hamper effective cleaning and sterilization. In one embodiment of the invention, wherein the mirror is connected inseparably to the handle this would be the case due to the constructed duct for the purpose of narrowing air density and due to the air guides in the compressed air duct. Therefore it is as part of the invention that a better cleanable and sterilizable composition of the handle and the mirror is developed, characterized by a protrusion that is inextricably attached to the mirror on the outflow side but at the inside of the air duct and which can be inserted detachably into the handle by clamping or otherwise releasably fastened means, and by being shaped three dimensionally in such a way that it the protrusion directly narrows the opening of the air outlet in such a way that the air flow will diverge just before the outflow opening to a downward effective flow on the mirror surface whereby the protrusion on itself from all sides is accessible for cleaning and the air duct in itself stays completely hollow.

Moreover this makes possible that several mirrors of a distinctive shape, dispersion angle and size of surface can be placed in the handle with maintenance of the characteristics of the invention, by means of the three-dimensional protrusion that is inextricably attached to each specific mirror.

In order to tacilitate the cleaning and sterilization of the handle and to make possible use of the mirror with handle without air supply, the link between air supply and the handle is arranged in such a way that the air supply pipe contains a rigid end part that only is to be inserted into the air duct at the back of the handle to enable coupling through the mutual magnetically attracted rings of which one is inextricably attached along the rigid end part of the air supply and the other is inextricably attached onto the hollow handle, thus providing an air-tight connection and by which the air duct in the handle and the air supply tube are leak free connected with each other and by which the magnetic adhesive force is chosen in such a way that the coupling is maintained when a pulling force is applied in a situation that the handle is normally used and that is disconnected by means of a larger exertion hand force applied to it.

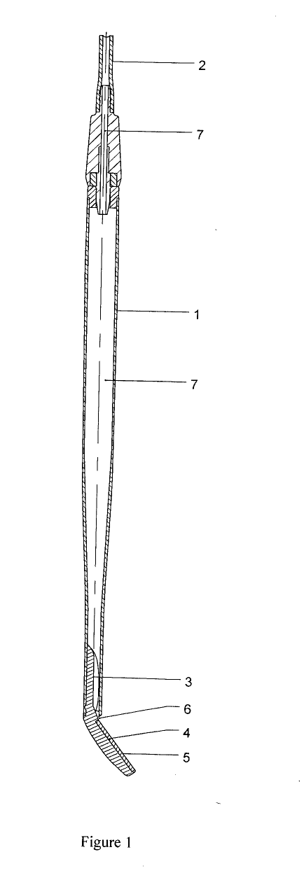

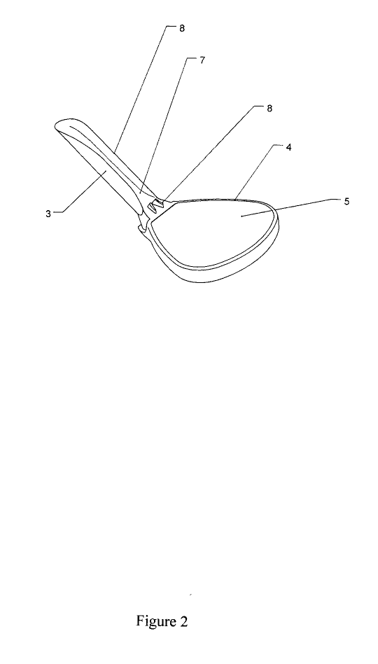

On the basis of FIGS. 1-5, the invention will in a preferred embodiment be described in detail. FIG. 1 shows a handle (1) in longitudinal cross-section with an air duct (7) and a flexible air supply pipe connected at the top (2) and inserted profusion at the bottom (3) connected with a mirror (4) with a hydrophobic and non-stick made reflecting surface (5) that is unlimited from the air outlet (6) of the air duct (7) to all directions of the reflecting surface (5) i.e. not limited by an upstanding casing or rim. FIG. 2 shows the protrusion (3) standing on its own with the mirror (4) that is rigidly attached to it. At the upper side of the protrusion (3) either vertical walls or control surfaces (8) are formed, that have been arranged into such an angle with respect to the longitudinal direction (i.e. in the air flow direction) of the protrusion (3) and in such way that the air flowing along those surfaces is diverged congruent to the at the protrusion connected angle of the mirror surface (5). The shape of the perimeter of the protrusion (3) is describing a peripheral portion of a cylinder of equal shape as the inner circumference of the air duct (7) and has an equal or fraction larger outer diameter, thus enabling the protrusion (3) when inserted into the duct (7) to clamp into said air duct (7) and the protrusion further has a wall thickness (9) arranged in such a way that it contains the function of progressively narrowing and flattening the air outlet (7) in the direction of the estuary in order to faster and under downward pressure diverge the air over the mirror surface (5)

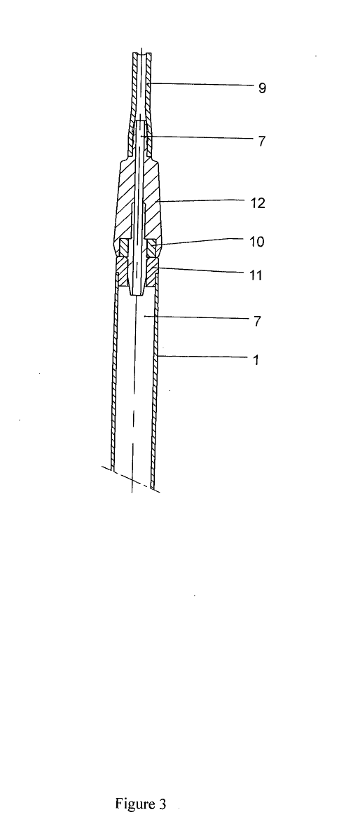

FIG. 3 shows a longitudinal section of the handle (1) coupled with the connected air hose (9) that at its other rear end is connected to an air compressor, which mutual coupling is formed by two mutually magnetically attracted rings (10) and (11), of which one ring (10) is inextricably attached around the hose or around a tube part (12) that is connected to the hose (12) and the other ring (11) is positioned at the top, inextricably attached to the handle (1) and placed around the air duct (7) is disposed , in such a way that the rings in connected state connect the air duct (7) with the hose leak-proof through an attractive force, which is greater than the possible force exerted thereon during normal use of the mirror and smaller than above hand pull force, so that connection can be easily broken by pulling apart the hose and the handle by hand force.

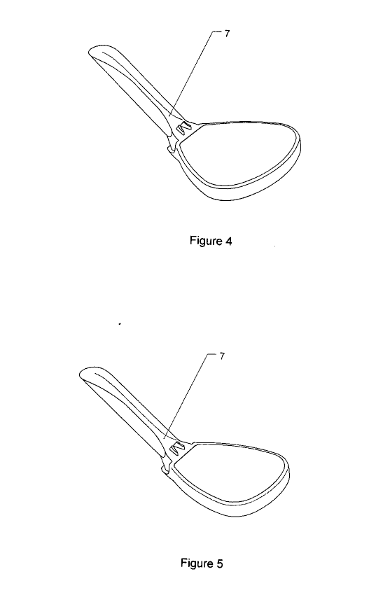

FIGS. 4 and 5 show two mirrors of different size and shape with a partial identically shaped part of protrusion , as far as it concerns the insertion in the air duct (7) and an different size and shape of the protrusion in so far as it concerns the control of the flow of air over the reflecting surface.

1. Method to keep the mirror surface of a dental mirror or mirror for medical probe with handle free of moisture and dirt during use through an compressed air flowing out of a duct that is shaped on or in the handle, further characterized in such a way that the mirror surface contains a hydrophobic and preferably, super-hydrophobic, and non-stick nature, created at any in the state of the art-known or yet to be known manner.

2. Method according to claim 1, with the characterization that the air flow is pressed from the air outlet of an air duct that is situated in or on the handle and in an angle of between 5 and 25 degrees from the side on to the reflecting surface and is spread over that mirror surface.

3. Method according to claim 1-2, characterized in that the mirror surface is not framed or cased and ends freely.

4. Method according to claims 1 and 2, characterized in that the duct is progressively narrowing and flattening to into the direction of the air outlet through which causing the air flow rate at the air outlet to increase.

5. Method according to claim 1-4, characterized in that the flow of air is controlled by means of upstanding control surfaces steering the air over the reflective surface.

6. Method according to claim 1-5, characterized in that the contour of the mirror is triangular and the air flow is compressed to blow onto the reflecting surface in an angle to the reflecting surface and controllably steered over the mirror surface by means of upright control surfaces positioned in the air duct in such a way that the air flows congruently diverging over the mirror surface in an angle congruent to the triangular shape of the mirror.

7. Mirror with handle for dental or medical examination, characterized in that it is equipped for applying the method as described in one or more of the preceding claims 2-6 and of claim 1.

8. A mirror as claimed in claims 5 to 7, characterized in that an inextricably to the mirror attached protrusion is inserted through the air outlet into the air duct in the handle and detachably clamped or in any other way again removably therein is attached, wherein said protrusion is three-dimensional shaped, in such a way that towards the air outlet the air duct is progressively narrowing the air way and flattening it in order to increase air pressure, in order to diverge at higher speeds from the opening of the air outlet on to the mirror surface whereas the protrusion itself is from all sides accessible for cleaning and the hollow duct is not distorted into an inaccessible duct.

9. A mirror as claimed in claims 7 and 8, characterized in that mirrors with a wide range of shapes and sizes are provided with a protrusion which fits into one and the same air duct and that are specifically shaped to enable cleaning of the mirror surface connected to that protrusion.

10. A mirror as claimed in claim 7-9, characterized in that the air duct passes through the handle and that the coupling of this air duct to a compressor-connected air hose consists of two mutual magnetically attracted rings, one of which is rigidly connected to a tube that is connected to the hose and the other is rigidly mounted in the air duct of the handle, which provides a leak-proof connection between the air duct, the handle and hose, whereby the magnetic attraction is chosen in such a way that the coupling is maintained at a normal use exerted traction and disconnected at greater exerted hand pull.

11. A mirror as claimed in claim 6-10, characterized in that the dental mirror can also be used separately from the air supply.
